(19) United States
(12) Patent Application Publication
(10) Pub. No.: US 2025/0073360 A1
(43) Pub. Date: Mar. 6, 2025

(54) Self-Disinfecting Disinfecting Systems and Methods

(71) Applicant: Bard Access Systems, Inc., Salt Lake City, UT (US)

(72) Inventors: Dustin Payne, West Jordan, UT (US); Ejaz S. Haque, Salt Lake City, UT (US)

(21) Appl. No.: 18/239,048

(22) Filed: Aug. 28, 2023

(51) Int. Cl. A61L 2/08; A61L 2/232; A61L 2/24

(52) U.S. Cl. CPC A61L 2/088; A61L 2/232; A61L 2/24; A61L 2202/11; A61L 2202/14; A61L 2202/25

(57) ABSTRACT

A self-disinfecting system can include a cladding having a flexible body that conforms to a substrate. The system can further include a disinfection means for disinfecting an exterior surface of the cladding. The disinfection means can be selected from a germicidal irradiation means, a photocatalytic disinfection means, or a chemical disinfection means for disinfecting the exterior surface of the cladding. The germicidal irradiation means can employ germicidal radiation from one or more locations within the body or the substrate to disinfect the cladding. The photocatalytic disinfection means can employ excitation radiation to produce reactive oxygen species using a photosensitizer incorporated into one or more polymeric layers of the body or a coating thereover to disinfect the cladding. The chemical disinfection means can employ a chemical disinfectant incorporated into the one-or-more polymeric layers of the body to disinfect the cladding.

### BACKGROUND

Various approaches have been developed for disinfecting surfaces, particularly in environments where cleanliness and hygiene are of utmost importance. Traditional methods of disinfection often involve the use of chemical disinfectants, such as sprays or wipes, which can be time-consuming and require repeated applications. Additionally, such chemical disinfectants can pose health risks or cause damage to the surfaces being treated. In recent years, there has been a growing interest in developing self-disinfecting systems that can continuously and effectively disinfect surfaces without the need for manual intervention.

Disclosed herein are self-disinfecting systems and methods in service of such a growing interest.

### SUMMARY

Disclosed herein is a self-disinfecting system including, in some embodiments, a cladding and a disinfection means for disinfecting an exterior surface of the cladding. The cladding includes a flexible body formed of one or more polymeric layers, which is configured to conform to a substrate. The disinfection means is selected from a germicidal irradiation means, a photocatalytic disinfection means, and a chemical disinfection means for disinfecting the exterior surface of the cladding. The germicidal irradiation means for disinfecting the exterior surface of the cladding employs irradiation with germicidal radiation from one or more locations within the body of the cladding, the substrate under the cladding, or the body of the cladding and the substrate under the cladding. The photocatalytic disinfection means for disinfecting the exterior surface of the cladding employs irradiation with excitation radiation from the one-or-more locations within the body of the cladding, the substrate under the cladding, or the body of the cladding and the substrate under the cladding. The excitation radiation is configured to produce reactive oxygen species (“ROS”) by way of a photosensitizer incorporated into the one-or-more polymeric layers of the body or a coating thereover. The chemical disinfection means for disinfecting the exterior surface of the cladding employs chemical disinfection with a chemical disinfectant incorporated into the one-or-more polymeric layers of the body.

In some embodiments, the body of the cladding is formed into a strip or sheet configured to conform to one or more contiguous surfaces of the substrate selected from flat and rounded surfaces of the substrate.

In some embodiments, the body of the cladding is formed into a sheath configured to conform to a cylindrical surface of the substrate.

In some embodiments, the germicidal irradiation means for disinfecting the exterior surface of the cladding includes one or more light emitters selected from light sources and optical-fiber termini operably connected to one or more of the light sources. The light sources include light-emitting diodes (“LEDs”), superluminescent LEDs (“SLEDs”), laser diodes, light bulbs, and tube lights.

In some embodiments, the one-or-more light emitters are configured to emit light selected from broad spectrum ultraviolet (“UV”) light, UVA light, UVB light, UVC light, blue light, and modulated light thereof. The modulated light is modulated with respect to frequency, power, duration, or a combination thereof.

In some embodiments, the one-or-more light emitters are within the body of the cladding between an outer polymeric layer and an inner polymeric layer, which correspond to at least two polymeric layers of the one-or-more polymeric layers. The outer polymeric layer of the body is transparent to the light emitted by the one-or-more light emitters.

In some embodiments, the one-or-more light emitters are within the substrate. Both the substrate and the one-or-more polymeric layers of the body of the cladding are transparent to the light emitted by the one-or-more light emitters.

In some embodiments, the photosensitizer is incorporated into the one-or-more polymeric layers of the body.

In some embodiments, the photosensitizer is incorporated into the coating over the one-or-more polymeric layers of the body.

In some embodiments, the photocatalytic disinfection means for disinfecting the exterior surface of the cladding includes one or more light emitters selected from light sources and optical-fiber termini operably connected to one or more of the light sources. The light sources include LEDs, SLEDs, laser diodes, light bulbs, and tube lights.

In some embodiments, the one-or-more light emitters are within the body of the cladding between an outer polymeric layer and an inner polymeric layer, which correspond to at least two polymeric layers of the one-or-more polymeric layers. The outer polymeric layer of the body is transparent to light emitted by the one-or-more light emitters.

In some embodiments, the one-or-more light emitters are within the substrate. Both the substrate and the one-or-more polymeric layers of the body of the cladding are transparent to light emitted by the one-or-more light emitters.

In some embodiments, the germicidal irradiation means or the photocatalytic disinfection means for disinfecting the exterior surface of the cladding includes electronic circuitry configured to power and control operation of the germicidal irradiation means or the photocatalytic disinfection means.

In some embodiments, the electronic circuitry includes one or more sensors configured to sense a person for starting or stopping disinfection of the exterior surface of the cladding by the germicidal irradiation means or the photocatalytic disinfection means upon sensing the person. The one-or-more sensors are selected from passive infrared sensors, ultrasonic sensors, microwave sensors, acoustic sensors, floor pressure mats, infrared beam sensors, capacitive proximity sensors, thermal cameras, laser sensors, radiofrequency (“RF”) sensors, and vibration sensors.

In some embodiments, the substrate is selected from a rail or frame of a hospital bed; a grab bar; an intravenous (“IV”) pole or stand; a surface or edge of an overbed table, a bedside table, a countertop, or a trash can; a sink, a faucet, or a toilet; a handle or knob of a door, a drawer, or a cabinet; a light switch, and a call button.

Also disclosed herein is a method of a self-disinfecting system. The method includes, in some embodiments, a disinfecting operation. The disinfecting operation includes disinfecting an exterior surface of a cladding conformed to a substrate. The disinfecting further includes a germicidal-irradiation operation of irradiating the exterior surface of the cladding with germicidal radiation from one or more locations within a flexible body of the cladding, the substrate under the cladding, or the body of the cladding and the substrate under the cladding. Alternatively, the disinfecting further includes an excitation-irradiation operation of irradiating the exterior surface of the cladding with excitation radiation from the one-or-more locations within the body of the cladding, the substrate under the cladding, or the body of the cladding and the substrate under the cladding. The excitation radiation produces ROS by way of a photosensitizer incorporated into one or more polymeric layers of the body or a coating thereover. Further alternatively, the disinfecting further includes a chemical-treatment operation of chemically treating the exterior surface of the cladding with a chemical disinfectant incorporated into the one-or-more polymeric layers of the body.

In some embodiments, the body of the cladding is formed into a strip or sheet configured to conform to one or more contiguous surfaces of the substrate selected from flat and rounded surfaces of the substrate.

In some embodiments, the body of the cladding is formed into a sheath configured to conform to a cylindrical surface of the substrate.

In some embodiments, the germicidal-irradiation operation or the excitation-irradiation operation of irradiating the exterior surface of the cladding includes irradiating with one or more light emitters selected from light sources and optical-fiber termini operably connected to one or more of the light sources. The light sources include LEDs, SLEDs, laser diodes, light bulbs, and tube lights. The one-or-more light emitters are within the substrate or the body of the cladding between an outer polymeric layer and an inner polymeric layer, which correspond to at least two polymeric layers of the one-or-more polymeric layers.

In some embodiments, the method further includes a sensing operation. The sensing operation includes sensing a person with one or more sensors of the self-disinfecting system. Additionally, the sensing operation includes starting or stopping the disinfecting of the exterior surface of the cladding in response to the person leaving or arriving, respectively. The one-or-more sensors are selected from passive infrared sensors, ultrasonic sensors, microwave sensors, acoustic sensors, floor pressure mats, infrared beam sensors, capacitive proximity sensors, thermal cameras, laser sensors, RF sensors, and vibration sensors.

In some embodiments, the substrate is selected from a rail or frame of a hospital bed; a grab bar; an IV pole or stand; a surface or edge of an overbed table, a bedside table, a countertop, or a trash can; a sink, a faucet, or a toilet; a handle or knob of a door, a drawer, or a cabinet; a light switch, and a call button.

These and other features of the concepts provided herein will become more apparent to those of skill in the art in view of the accompanying drawings and following description, which describe particular embodiments of such concepts in greater detail.

### DRAWINGS

FIG. 1 illustrates a self-disinfecting system in accordance with some embodiments.

FIG. 2 illustrates a detailed view of a cladding of the self-disinfecting system conforming to a substrate in accordance with some embodiments.

FIG. 3 illustrates a body of the cladding formed into a sheath in accordance with some embodiments.

FIG. 4 illustrates the body of the cladding formed into a sheet in accordance with some embodiments.

FIG. 5 illustrates a longitudinal cross section of the cladding conforming to the substrate of FIG. 2, wherein one or more light emitters are within the substrate in accordance with some embodiments.

FIG. 6 illustrates a longitudinal cross section of the cladding, wherein the one-or-more light emitters are within the body of the cladding in accordance with some embodiments.

FIG. 7 illustrates production of disinfecting ROS upon irradiation of the cladding having a photosensitizer incorporated therein in accordance with some embodiments.

FIG. 8 illustrates electronic circuitry configured to power and control operation of the self-disinfecting system in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

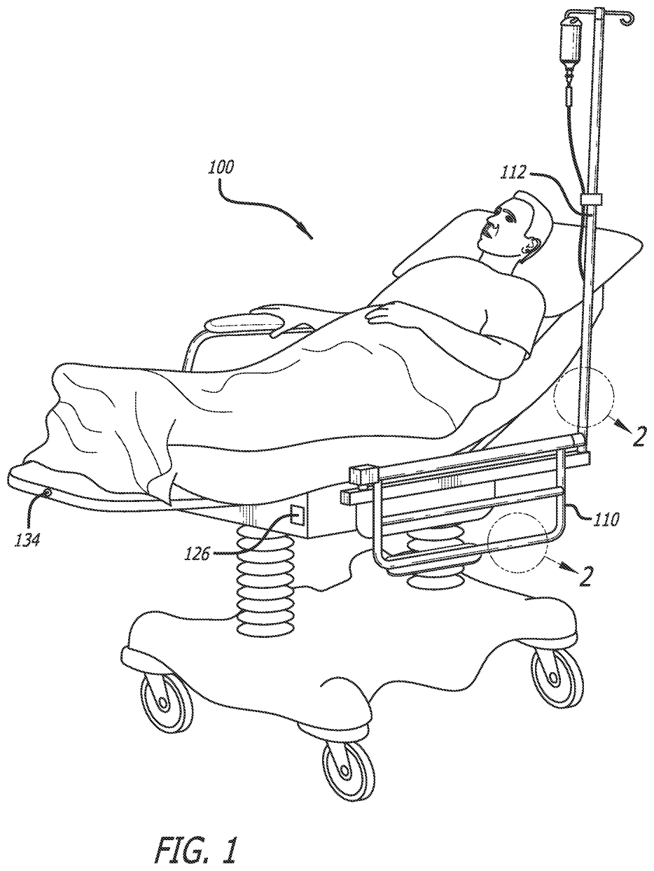

FIG. 1 illustrates a self-disinfecting system 100 in accordance with some embodiments.

As shown, the self-disinfecting system 100 can include a cladding 102 and a disinfection means for disinfecting an exterior surface 104 of the cladding 102.

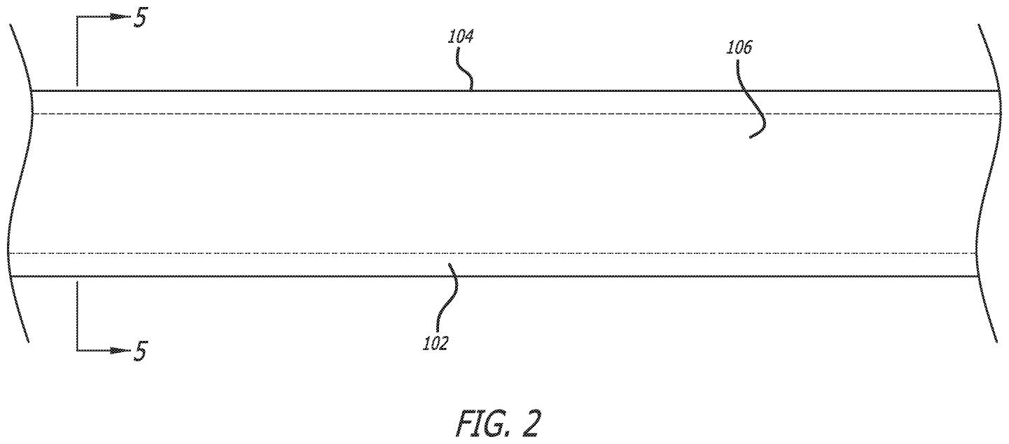

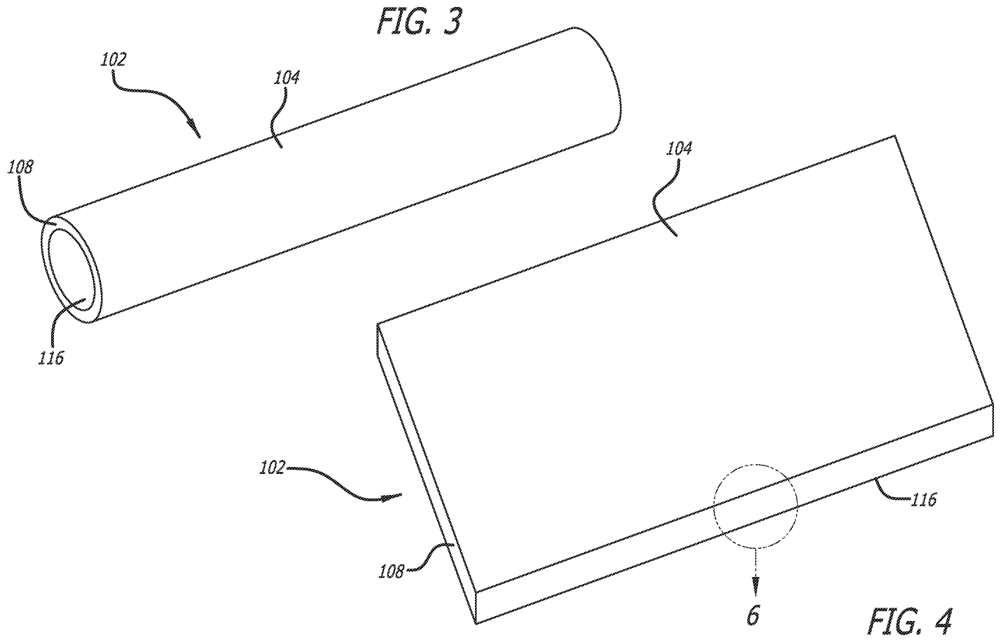

FIG. 2 illustrates the cladding 102 of the self-disinfecting system 100 conforming to a substrate 106 in accordance with some embodiments. FIGS. 3 and 4 illustrate a flexible body 108 of the cladding 102 respectively formed into a sheath and sheet in accordance with some embodiments.

The cladding 102 includes the body 108 configured to conform to the substrate 106. Being as the substrate 106 can be any high-contact or contamination-prone structure selected from a rail or frame of a hospital bed such as rail 110; a support-providing grab bar; an IV pole or stand such as IV pole 112; a surface or edge of an overbed table, a bedside table, a countertop, or a trash can; a sink, a faucet, or a toilet; a handle or knob of a door, a drawer, or a cabinet; a light switch, and a call button, the body 108 can be in any of various forms for conforming to the substrate 106. In an example, the body 108 of the cladding 102 can be formed into a strip or sheet configured to conform to one or more contiguous surfaces of the substrate 106, wherein the one-or-more surfaces are selected from flat and rounded surfaces of the substrate 106. The flat surfaces of the substrate 106 can be any top or side surfaces of the foregoing overbed table, bedside table, countertop, or trash, and the rounded surfaces can be any rounded edges joining the top or side surfaces. In another example, the body 108 of the cladding 102 can be formed into a sheath configured to conform to a cylindrical surface of the substrate 106. The cylindrical surface of the substrate 106 can be that of a circular or elliptical cylinder such as the rail 110; however, the body 108 of the cladding 102 can be formed into a sheath configured to conform to any elongate shape having a traverse cross section corresponding to a geometric shape including at least a simple convex polygon.

The body 108 of the cladding 102 can be formed of one or more polymeric layers 114. When the body 108 is formed a plurality of polymeric layers 114, any two of the polymeric layers 114 can be independently joined together by calendaring, lamination, heat welding, ultrasonic welding, solvent bonding, adhesive bonding, RF welding, thermal bonding, co-extrusion, or injection overmolding. Notably, any polymeric layer(s) of the one-or-more polymeric layers 114 up to an entirety of the body 108 can be formed of a polymeric material (e.g., TOPAS® cyclic olefin copolymer by TOPAS of Raunheim, Germany) with a thickness sufficient for structural integrity of the polymeric layer(s) 114 or body 108 and a transparency sufficient for the light emitted by the one-or-more light emitters 118 to pass therethrough. Whether the body 108 is formed of one polymeric layer 114 or the plurality of polymeric layers 114, the body 108 includes the exterior surface 104 and an interior surface 116 opposite the exterior surface 104, each surface of the exterior and interior surfaces 104 and 116 being coextensive with those of the cladding 102 itself. Notably, the interior surface 116 of the cladding 102 can include an adhesive thereon configured to adhere the cladding 102 to the one-or-more surfaces of the substrate 106.

The disinfection means is selected from a germicidal irradiation means, a photocatalytic disinfection means, and a chemical disinfection means for disinfecting the exterior surface 104 of the cladding 102. Being as the cladding 102 can vary in accordance with the germicidal irradiation means, the photocatalytic disinfection means, and the chemical disinfection means, further description of the cladding 102 is set forth below in the description for the disinfection means.

The germicidal irradiation means for disinfecting the exterior surface 104 of the cladding 102 can irradiate at least the exterior surface 104 of the cladding 102 with germicidal radiation including light selected from broad spectrum UV light, UVA light, UVB light, UVC light, blue light, and modulated light thereof, for example, light modulated with respect to wavelength or frequency, power, including ramping the power, duration, including pulse durations when pulsing the modulated light, or a combination thereof. Such light can be emitted from one or more light emitters 118 selected from light sources 119 and optical-fiber termini operably connected to one or more of the light sources 119. The light sources 119 can include LEDs, SLEDs, laser diodes, light bulbs, and tube lights.

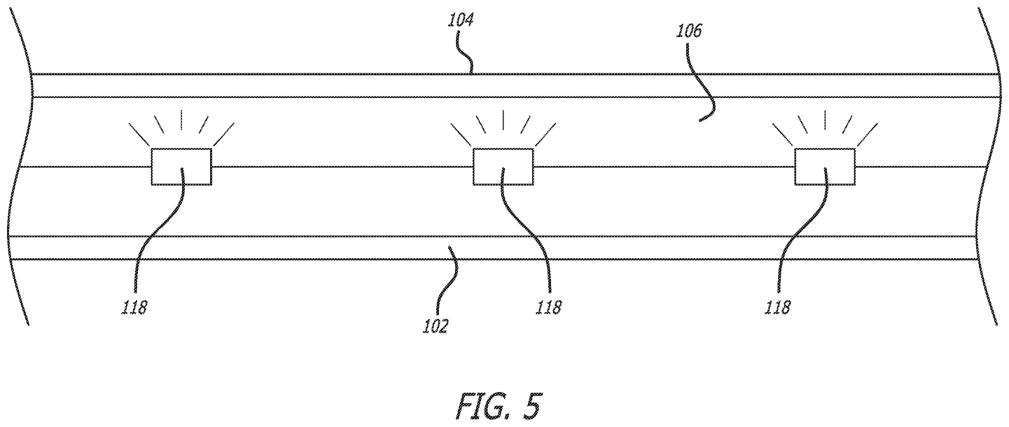

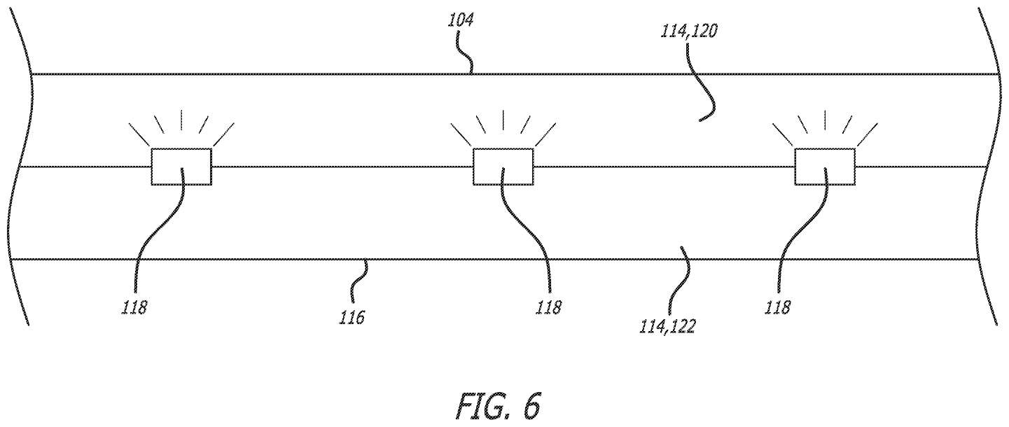

FIG. 5 illustrates a longitudinal cross section of the cladding 102 conforming to the substrate 106 of FIG. 2, wherein the one-or-more light emitters 118 are within the substrate 106 in accordance with some embodiments. FIG. 6 illustrates a longitudinal cross section of the cladding 102, wherein the one-or-more light emitters 118 are within the body 108 of the cladding 102 in accordance with some embodiments.

The germicidal irradiation means for disinfecting the exterior surface 104 of the cladding 102 can irradiate at least the exterior surface 104 of the cladding 102 with the one-or-more light emitters 118 from one or more locations within the body 108 of the cladding 102, under or within the substrate 106 under the cladding 102, or within the body 108 of the cladding 102 and under or within the substrate 106 under the cladding 102.

As to the one-or-more light emitters 118 being within the body 108 of the cladding 102, the one-or-more light emitters 118 can be between an outer polymeric layer 120 and an inner polymeric layer 122 joined together as set forth above, wherein the outer polymeric layer 120 and the inner polymeric layer 122 correspond to at least two polymeric layers of the plurality of polymeric layers 114. Further, the one-or-more light emitters 118 can be arranged to emit light toward the outer polymeric layer 120 and, thus, the exterior surface 104 of the cladding 102. At least the outer polymeric layer 120 of the body 108 is sufficiently transparent to the light emitted by the one-or-more light emitters 118 to allow the germicidal radiation to disinfect the exterior surface 104 of the cladding 102. Further, the outer polymeric layer 120 of the body 108 can be configured to diffuse the light emitted by the one-or-more light emitters 118 to allow the germicidal radiation to uniformly disinfect the exterior surface 104 of the cladding 102.

As to the one-or-more light emitters 118 being under or within the substrate 106, the one-or-more light emitters 118 can be arranged to emit light from the substrate 106 toward the cladding 102 and, thus, the exterior surface 104 of the cladding 102. Both the substrate 106 and the body 108 of the cladding 102 are sufficiently transparent to the light emitted by the by the one-or-more light emitters 118 to allow the germicidal radiation to disinfect the exterior surface 104 of the cladding 102. Further, the substrate 106, the body 108 of the cladding 102, or both the substrate 106 and the body 108 of the cladding 102 can be configured to diffuse the light emitted by the one-or-more light emitters 118 to allow the germicidal radiation to uniformly disinfect the exterior surface 104 of the cladding 102.

The photocatalytic disinfection means for disinfecting the exterior surface 104 of the cladding 102 can irradiate at least the exterior surface 104 of the cladding 102 with excitation radiation including light selected from broad spectrum UV-vis light, broad spectrum UV light, UVA light, UVB light, UVC light, broad spectrum visible light, violet light, blue light, green light, yellow light, orange light, red light, or modulated light thereof, for example, light modulated with respect to wavelength or frequency, power, including ramping the power, duration, including pulse durations when pulsing the modulated light, or a combination thereof. Such light can be emitted from the one-or-more light emitters 118 set forth above, namely those selected from the light sources 119 and the optical-fiber termini operably connected thereto, the light sources 119 including LEDs, SLEDs, laser diodes, light bulbs, and tube lights.

The photocatalytic disinfection means for disinfecting the exterior surface 104 of the cladding 102 can irradiate at least the exterior surface 104 of the cladding 102 with the one-or-more light emitters 118 from the one-or-more locations within the body 108 of the cladding 102, under or within the substrate 106 under the cladding 102, or within the body 108 of the cladding 102 and under or within the substrate 106 under the cladding 102.

As to the one-or-more light emitters 118 being within the body 108 of the cladding 102, the one-or-more light emitters 118 can be between the outer polymeric layer 120 and the inner polymeric layer 122 joined together as set forth above, wherein the outer polymeric layer 120 and the inner polymeric layer 122 correspond to at least two polymeric layers of the plurality of polymeric layers 114. Further, the one-or-more light emitters 118 can be arranged to emit light toward the outer polymeric layer 120 and, thus, the exterior surface 104 of the cladding 102. At least the outer polymeric layer 120 of the body 108 is sufficiently transparent to the light emitted by the one-or-more light emitters 118 to allow the excitation radiation to produce ROS by way of the photosensitizer 124 incorporated (e.g., blended as a filler) into the outer polymeric layer 120 or the coating thereover to disinfect the exterior surface 104 of the cladding 102. Further, the outer polymeric layer 120 of the body 108 can be configured to diffuse the light emitted by the one-or-more light emitters 118 to allow the excitation radiation to uniformly produce the ROS in the outer polymeric layer 120 or the coating thereover to disinfect the exterior surface 104 of the cladding 102 with the ROS.

As to the one-or-more light emitters 118 being under or within the substrate 106, the one-or-more light emitters 118 can be arranged to emit light from the substrate 106 toward the cladding 102 and, thus, the exterior surface 104 of the cladding 102. Both the substrate 106 and the body 108 of the cladding 102 are sufficiently transparent to the light emitted by the one-or-more light emitters 118 to allow the excitation radiation to produce the ROS in the body 108 of the cladding 102 or the coating thereover to disinfect the exterior surface 104 of the cladding 102. Further, the substrate 106, the body 108 of the cladding 102, or both the substrate 106 and the body 108 of the cladding 102 can be configured to diffuse the light emitted by the one-or-more light emitters 118 to allow the excitation radiation to uniformly produce the ROS in the body 108 of the cladding 102 or the coating thereover to disinfect the exterior surface 104 of the cladding 102 with the ROS.

Referring again to the body 108 of the cladding 102 and the one-or-more polymeric layers 114 thereof, a photosensitizer 124 can be incorporated (e.g., blended as a filler) into any polymeric layer of the one-or-more polymeric layers 114. Additionally or alternatively, the photosensitizer 124 can be similarly incorporated into a coating (e.g., a polymeric coating) over the one-or-more polymeric layers 114. Regardless, the photosensitizer 124 should be incorporated into the body 108 of the cladding 102 outward from the one-or-more light emitters 118. In other words, the photosensitizer 124 should be incorporated into the body 108 of the cladding 102 such that the polymeric layer(s) 114 or coating including the photosensitizer 124 is between an agent of touch contamination (e.g., patient, clinician, visitor, etc.) and the one-or-more light emitters 118.

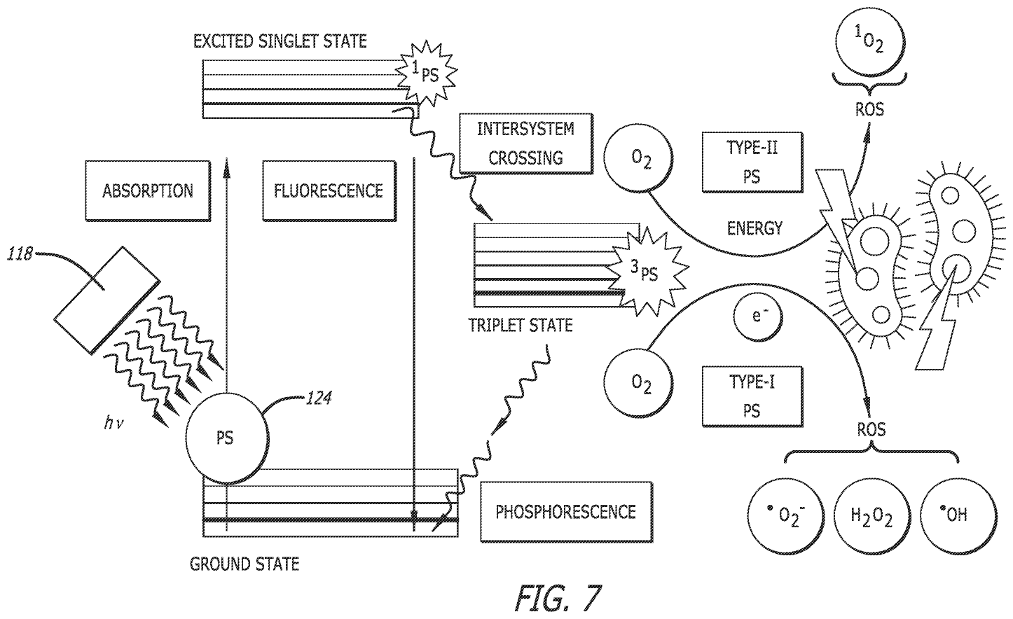

FIG. 7 illustrates production of the ROS upon irradiation of the cladding 102 having the photosensitizer 124 incorporated therein in accordance with some embodiments.

The excitation radiation is configured to produce the ROS by way of the photosensitizer 124 incorporated into the one-or-more polymeric layers 114 of the body 108 or the coating thereover. As shown, upon irradiation with the excitation radiation, the photosensitizer 124, or some population of the photosensitizer 124, enters an excited singlet state. While the photosensitizer 124 can lose energy by way of fluorescence, some population of the photosensitizer 124 in the excited single state alternatively enters an excited triplet state through intersystem crossing. And, depending upon whether the photosensitizer 124 is a type-I photosensitizer or a type-II photosensitizer, the photosensitizer 124 in its excited triplet state reacts with oxygen by way of electron transfer or energy transfer to produce the ROS for disinfecting the exterior surface 104 of the cladding 102. Indeed, the type-I photosensitizer in its excited triplet state reacts with oxygen by way of electron transfer to produce superoxide (•O₂⁻) by reduction of oxygen, hydrogen peroxide (H₂O₂) by disproportionation of the superoxide, and hydroxyl radical (•OH) by reduction of the hydrogen peroxide. The type-II photosensitizer in its excited triplet state reacts with oxygen by way of energy transfer to produce singlet oxygen (102). The ROS, in turn, lead to microbial death and inactivation of microbial endotoxins by various reaction mechanisms involving the ROS.

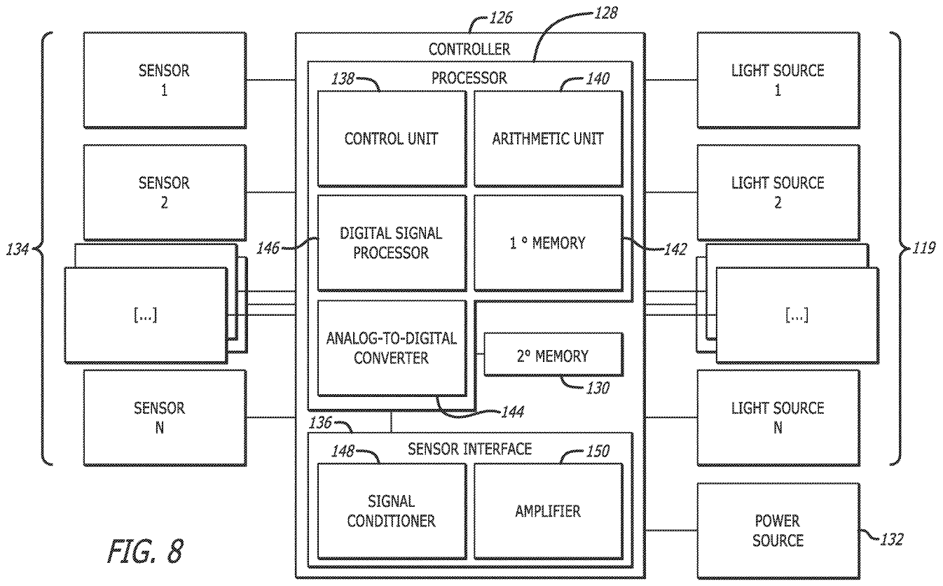

FIG. 8 illustrates electronic circuitry configured to power and control operation of the self-disinfecting system 100 in accordance with some embodiments.

The germicidal irradiation means or the photocatalytic disinfection means for disinfecting the exterior surface 104 of the cladding 102 can include electronic circuitry configured to power and control operation of the germicidal irradiation means or the photocatalytic disinfection means.

The electronic circuitry can include at least a controller 126 having a processor 128 and secondary memory 130, the light sources 119, and a power source 132. In addition, the electronic circuitry can include one or more sensors 134 configured to sense an agent of touch contamination (e.g., patient, clinician, visitor, etc.) for starting or stopping disinfection of the exterior surface 104 of the cladding 102 by the germicidal irradiation means or the photocatalytic disinfection means upon sensing the person. When the one-or-more sensors 134 are present, the controller 126 can further include a sensor interface 136. When the one-or-more sensors 134 are absent, the electronic circuitry can include a switch for switching the one-or-more light sources 119 on or off. Lastly, the electronic circuitry can include any requisite electrical leads between such electronic components of the electronic circuitry for powering and operating the germicidal irradiation means or the photocatalytic disinfection means.

The processor 128 can include a control unit 138, an arithmetic unit 140, and primary memory 142 (e.g., cache memory, random-access memory [“RAM”], or both), wherein the primary memory 142 can be configured to store in-use programs and data (e.g., the sensor data). While the primary memory 142 can be within a same package as a remainder of the processor 128 as alluded to in FIG. 8, at least the foregoing RAM can be distributed outside the package of the processor 128, for example, in its own package. In consideration of the one-or-more sensors 134 being analog sensors, to which the one-or-more sensors 134 need not be limited, the processor 128 can further include an analog-to-digital converter (“ADC”) 144 configured to convert electrical signals from the one-or-more sensors 134 from analog to digital and a digital-signal processor (“DSP”) 146 configured to generate sensor data from the electrical signals. While the ADC 144 and DSP 146 and can be within a same package as a remainder of the processor 128 as alluded to in FIG. 8, the ADC 144 and DSP 146 and can be distributed outside the package of the processor 128, for example, in their own package.

The secondary memory 130 can be configured to store data (e.g., the sensor data) and programs including instructions, logic including sensing logic, algorithms including sensing algorithms, or some combination thereof for loading into the primary memory 142 for use by the processor 128, for example, when determining from the sensor data in a sensing operation whether a person is within range of the self-disinfecting system 100. Notably, such a sensing operation can include starting or stopping the disinfecting of the exterior surface 104 of the cladding 102 in response to an agent of touch contamination (e.g., patient, clinician, visitor, etc.) respectively leaving or arriving at the self-disinfecting system 100 or a portion thereof.

The sensor interface 136 can include a signal conditioner 148 configured to standardize the electrical signals from the one-or-more sensors 134 through voltage or current limiting, anti-aliasing filtering, or the like. In addition, the sensor interface 136 can include an amplifier 150 configured to amplify the electrical signals and, thereby, increase their signal-to-noise ratio.

The one-or-more sensors 134 can be selected from passive infrared sensors, ultrasonic sensors, microwave sensors, acoustic sensors, floor pressure mats, infrared beam sensors, capacitive proximity sensors, thermal cameras, laser sensors, RF sensors, and vibration sensors.

The power source 132 can be configured to power the controller 126, the light sources 119, and the one-or-more sensors 134, if present. Such a power source 132 can be an external power source such as utility power or an internal power source including an internal battery, which internal battery can be rechargeable by way of a port.

The chemical disinfection means for disinfecting the exterior surface 104 of the cladding 102 employs chemical disinfection with a chemical disinfectant incorporated into the one-or-more polymeric layers 114 of the body 108 or a coating (e.g., polymeric coating) thereover. Indeed, like the photosensitizer 124, the chemical disinfectant can be incorporated (e.g., blended as a filler, covalently bonded, etc.) into the outer polymeric layer 120 or the coating thereover to disinfect the exterior surface 104 of the cladding 102. Such a chemical disinfectant can include one or more antimicrobial metals or their ions (via salts) selected from at least as copper, silver, and zinc; one or more antimicrobial metal compounds selected from at least copper oxide, silver oxide, zinc oxide, and titanium dioxide; one or more antimicrobial nanoparticles selected from at least copper nanoparticles, copper-oxide nanoparticles, silver nanoparticles, zinc-oxide nanoparticles, titanium-dioxide nanoparticles, gold nanoparticles, iron-oxide nanoparticles, cerium-oxide nanoparticles, magnesium-oxide nanoparticles, functionalized silica nanoparticles, chitosan nanoparticles, graphene nanoparticles, and graphene-oxide nanoparticles; one or more quaternary ammonium salts selected from at least benzalkonium chloride, cetrimonium bromide, and an alkyl dimethyl benzyl ammonium chloride; one or more pyridinium salts selected from cetylpyridinium chloride, laurylpyridinium chloride, myristylpyridinium chloride, octylpyridinium chloride, and dodecylpyridinium chloride; a guanidine-based antimicrobial selected from at least guanidine hydrochloride, a biguanide such as chlorhexidine, and polyhexamethylene biguanide, or some combination selected from the foregoing. Notably, certain antimicrobials serving as the chemical disinfectant benefit from activation by light (e.g., titanium dioxide by UV light), thereby providing another disinfection means for disinfecting the exterior surface 104 of the cladding 102.

### Methods

Methods include methods of the method of the self-disinfecting system 100. For example, a method of the self-disinfecting system 100 can include a disinfecting operation and, optionally, a sensing operation.

The disinfecting operation can include disinfecting the exterior surface 104 of the cladding 102 while the cladding 102 is conformed to the substrate 106. When the self-disinfecting system 100 includes the germicidal irradiation means for disinfecting the exterior surface 104 of the cladding 102, the disinfecting operation can further include a germicidal-irradiation operation of irradiating the exterior surface 104 of the cladding 102 with germicidal radiation from the one-or-more locations within the body 108 of the cladding 102, the substrate 106 under the cladding 102, or the body 108 of the cladding 102 and the substrate 106 under the cladding 102. When the self-disinfecting system 100 includes the photocatalytic means for disinfecting the exterior surface 104 of the cladding 102, the disinfecting operation can further include an excitation-irradiation operation of irradiating the exterior surface 104 of the cladding 102 with excitation radiation from the one-or-more locations within the body 108 of the cladding 102, the substrate 106 under the cladding 102, or the body 108 of the cladding 102 and the substrate 106 under the cladding 102. As set forth above, the excitation radiation produces the ROS by way of the photosensitizer 124 incorporated into the one-or-more polymeric layers 114 of the body 108 or the coating thereover. Lastly, when the self-disinfecting system 100 includes the chemical disinfection means for disinfecting the exterior surface 104 of the cladding 102, the disinfecting operation can further include a chemical-treatment operation of chemically treating the exterior surface 104 of the cladding 102 with the chemical disinfectant incorporated into the one-or-more polymeric layers 114 of the body 108.

The sensing operation can include sensing an agent of touch contamination (e.g., patient, clinician, visitor, etc.) with the one-or-more sensors 134 of the self-disinfecting system 100. Additionally, the sensing operation can include starting or stopping the disinfecting of the exterior surface 104 of the cladding 102 in response to the agent of touch contamination leaving or arriving, respectively.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures can be made from the particular embodiments disclosed herein without departing from the scope of the concepts provided herein.

What is claimed is:

1. A self-disinfecting system, comprising:
a cladding including a flexible body formed of one or more polymeric layers, the cladding configured to conform to a substrate; and
a disinfection means for disinfecting an exterior surface of the cladding, the disinfection means selected from:
a germicidal irradiation means for disinfecting the exterior surface of the cladding by irradiation with germicidal radiation from one or more locations within the body of the cladding, the substrate under the cladding, or the body of the cladding and the substrate under the cladding;
a photocatalytic disinfection means for disinfecting the exterior surface of the cladding by irradiation with excitation radiation from the one-or-more locations within the body of the cladding, the substrate under the cladding, or the body of the cladding and the substrate under the cladding, the excitation radiation configured to produce reactive oxygen species (“ROS”) by way of a photosensitizer incorporated into the one-or-more polymeric layers of the body or a coating thereover; and
a chemical disinfection means for disinfecting the exterior surface of the cladding by chemical disinfection with a chemical disinfectant incorporated into the one-or-more polymeric layers of the body.

2. The self-disinfecting system of claim 1, wherein the body of the cladding is formed into a strip or sheet configured to conform to one or more contiguous surfaces of the substrate selected from flat and rounded surfaces of the substrate.

3. The self-disinfecting system of claim 1, wherein the body of the cladding is formed into a sheath configured to conform to a cylindrical surface of the substrate.

4. The self-disinfecting system of claim 1, wherein the germicidal irradiation means for disinfecting the exterior surface of the cladding includes one or more light emitters selected from light sources including light-emitting diodes (“LEDs”), superluminescent LEDs (“SLEDs”), laser diodes, light bulbs, and tube lights, and optical-fiber termini operably connected to one or more of the light sources.

5. The self-disinfecting system of claim 4, wherein the one-or-more light emitters are configured to emit light selected from broad spectrum ultraviolet (“UV”) light, UVA light, UVB light, UVC light, blue light, and modulated light thereof, the modulated light being modulated with respect to frequency, power, duration, or a combination thereof.

6. The self-disinfecting system of claim 5, wherein the one-or-more light emitters are within the body of the cladding between an outer polymeric layer and an inner polymeric layer corresponding to at least two polymeric layers of the one-or-more polymeric layers, the outer polymeric layer of the body transparent to the light emitted by the one-or-more light emitters.

7. The self-disinfecting system of claim 5, wherein the one-or-more light emitters are within the substrate, both the substrate and the one-or-more polymeric layers of the body of the cladding transparent to the light emitted by the one-or-more light emitters.

8. The self-disinfecting system of claim 1, wherein the photosensitizer is incorporated into the one-or-more polymeric layers of the body.

9. The self-disinfecting system of claim 1, wherein the photosensitizer is incorporated into the coating over the one-or-more polymeric layers of the body.

10. The self-disinfecting system of claim 1, wherein the photocatalytic disinfection means for disinfecting the exterior surface of the cladding includes one or more light emitters selected from light sources including light-emitting diodes (“LEDs”), superluminescent LEDs (“SLEDs”), laser diodes, light bulbs, and tube lights, and optical-fiber termini operably connected to one or more of the light sources.

11. The self-disinfecting system of claim 10, wherein the one-or-more light emitters are within the body of the cladding between an outer polymeric layer and an inner polymeric layer corresponding to at least two polymeric layers of the one-or-more polymeric layers, the outer polymeric layer of the body transparent to light emitted by the one-or-more light emitters.

12. The self-disinfecting system of claim 10, wherein the one-or-more light emitters are within the substrate, both the substrate and the one-or-more polymeric layers of the body of the cladding transparent to light emitted by the one-or-more light emitters.

13. The self-disinfecting system of claim 1, wherein the germicidal irradiation means or the photocatalytic disinfection means for disinfecting the exterior surface of the cladding includes electronic circuitry configured to power and control operation of the germicidal irradiation means or the photocatalytic disinfection means.

14. The self-disinfecting system of claim 13, wherein the electronic circuitry includes one or more sensors configured to sense a person for starting or stopping disinfection of the exterior surface of the cladding by the germicidal irradiation means or the photocatalytic disinfection means upon sensing the person, the one-or-more sensors selected from passive infrared sensors, ultrasonic sensors, microwave sensors, acoustic sensors, floor pressure mats, infrared beam sensors, capacitive proximity sensors, thermal cameras, laser sensors, radiofrequency (“RF”) sensors, and vibration sensors.

15. The self-disinfecting system of claim 1, wherein the substrate is selected from a rail or frame of a hospital bed; a grab bar; an intravenous (“IV”) pole or stand; a surface or edge of an overbed table, a bedside table, a countertop, or a trash can; a sink, a faucet, or a toilet; a handle or knob of a door, a drawer, or a cabinet; a light switch, and a call button.

16. A method of a self-disinfecting system, comprising:
disinfecting an exterior surface of a cladding conformed to a substrate, the disinfecting including:
irradiating the exterior surface of the cladding with germicidal radiation from one or more locations within a flexible body of the cladding, the substrate under the cladding, or the body of the cladding and the substrate under the cladding;
irradiating the exterior surface of the cladding with excitation radiation from the one-or-more locations within the body of the cladding, the substrate under the cladding, or the body of the cladding and the substrate under the cladding, the excitation radiation producing reactive oxygen species (“ROS”) by way of a photosensitizer incorporated into one or more polymeric layers of the body or a coating thereover; or
chemically treating the exterior surface of the cladding with a chemical disinfectant incorporated into the one-or-more polymeric layers of the body.

17. The method of claim 16, wherein the body of the cladding is formed into a strip or sheet configured to conform to one or more contiguous surfaces of the substrate selected from flat and rounded surfaces of the substrate.

18. The method of claim 16, wherein the body of the cladding is formed into a sheath configured to conform to a cylindrical surface of the substrate.

19. The method of claim 16, wherein irradiating the exterior surface of the cladding includes irradiating with one or more light emitters selected from light sources including light-emitting diodes (“LEDs”), superluminescent LEDs (“SLEDs”), laser diodes, light bulbs, and tube lights, and optical-fiber termini operably connected to one or more of the light sources, the one-or-more light emitters within the substrate or the body of the cladding between an outer polymeric layer and an inner polymeric layer corresponding to at least two polymeric layers of the one-or-more polymeric layers.

20. The method of claim 16, the method further comprising:
sensing a person with one or more sensors of the self-disinfecting system; and
starting or stopping the disinfecting of the exterior surface of the cladding in response to the person leaving or arriving, respectively, the one-or-more sensors selected from passive infrared sensors, ultrasonic sensors, microwave sensors, acoustic sensors, floor pressure mats, infrared beam sensors, capacitive proximity sensors, thermal cameras, laser sensors, radiofrequency (“RF”) sensors, and vibration sensors.

21. The method of claim 16, wherein the substrate is selected from a rail or frame of a hospital bed; a grab bar; an intravenous (“IV”) pole or stand; a surface or edge of an overbed table, a bedside table, a countertop, or a trash can; a sink, a faucet, or a toilet; a handle or knob of a door, a drawer, or a cabinet; a light switch, and a call button.
